(19) United States
(12) Patent Application Publication
(10) Pub. No.: US 2024/0327496 A1
(43) Pub. Date: Oct. 3, 2024

(54) Improved Expression Vectors and Uses Thereof

(71) Applicant: Pfizer Inc., New York, NY (US)

(72) Inventors: Kalpanie Ruwanmali Bandara, North Reading, MA (US); Kathryn Mary Beal, Londonderry, NH (US); John Joseph Scarcelli, Holliston, MA (US); Lin Zhang, Basking Ridge, NJ (US)

(73) Assignee: Pfizer Inc., New York, NY (US)

(21) Appl. No.: 18/293,889

(22) PCT Filed: Jul. 29, 2022
(86) PCT No.: PCT/IB2022/057073

§ 371 Date: Jan. 31, 2024

Related U.S. Application Data
(60) Provisional application No. 63/228,315, filed on Aug. 2, 2021.

(51) Int. Cl. C07K 16/00; C12N 15/85

(52) U.S. Cl. CPC C07K 16/00; C12N 15/85; C07K 2317/14; C07K 2319/00; C12N 2800/30

(57) ABSTRACT

Vectors and nucleic acid constructs for improved antibody production are provided. Also provided are methods of making and using the vectors and constructs.

### FIELD

Provided herein are improved protein expression vectors, cells containing the vectors, and related compositions and methods. The expression vectors may be used for expression of proteins such as antibodies in host cells.

### BACKGROUND

Recombinant antibodies and related molecules (e.g. antibody fusion proteins) are important for multiple uses, such as medical therapeutics and diagnostics. While effective methods exist for the production of recombinant antibodies, production of recombinant antibodies remains a complicated and expensive process.

Accordingly, there is a need for compositions and methods that would improve the quantity and/or quality of recombinant antibodies generated during recombinant antibody production.

### SUMMARY

The present invention relates to improved protein expression vectors, cells containing the vectors, and related compositions and methods. The expression vectors may be used for expression of proteins such as antibodies in host cells.

In some embodiments, provided herein is a mammalian host cell comprising a first exogenous nucleic acid construct integrated into a host cell chromosome and a second exogenous nucleic acid construct integrated into a host cell chromosome, wherein the first exogenous nucleic acid construct comprises in 5′ to 3′ order a first gene encoding an antibody light chain and a second gene encoding an antibody light chain, and wherein the second exogenous nucleic acid construct comprises in 5′ to 3′ order a first gene encoding an antibody heavy chain or antibody heavy chain fusion protein and a second gene encoding an antibody heavy chain.

In some embodiments, provided herein is a mammalian host cell comprising a first exogenous nucleic acid construct integrated into a host cell chromosome and a second exogenous nucleic acid construct integrated into a host cell chromosome, wherein the first exogenous nucleic acid construct comprises the following elements in 5′ to 3′ order: a) first recombination sequence; b) first gene encoding an antibody light chain; c) second gene encoding an antibody light chain; d) second recombination sequence, and wherein the second exogenous nucleic acid construct comprises the following elements in 5′ to 3′ order: a) first recombination sequence; b) first gene encoding an antibody heavy chain or antibody heavy chain fusion protein; c) second gene encoding an antibody heavy chain; d) second recombination sequence.

In a host cell provided herein, in some embodiments, the second exogenous nucleic acid construct first gene encodes an antibody heavy chain. In some embodiments, the second exogenous nucleic acid construct first gene encodes an antibody heavy chain fusion protein. In some embodiments, the first recombination sequence of the first exogenous nucleic acid construct has a different nucleotide sequence than the second recombination sequence of the first exogenous nucleic acid construct by at least one nucleotide. In some embodiments, the first recombination sequence of the first exogenous nucleic acid construct and the first recombination sequence of the second exogenous nucleic acid construct are recognized by the same recombinase enzyme. In some embodiments, the first recombination sequence of the first exogenous nucleic acid construct, the second recombination sequence of the first exogenous nucleic acid construct, the first recombination sequence of the second exogenous nucleic acid construct, and the second recombination sequence of the second exogenous nucleic acid construct are recognized by the same recombinase enzyme.

In a host cell provided herein, in some embodiments, the first recombination sequence of the first exogenous nucleic acid construct has the same nucleotide sequence as the first recombination sequence of the second exogenous nucleic acid construct. In some embodiments, the first recombination sequence of the first exogenous nucleic acid construct has a different nucleotide sequence than the first recombination sequence of the second exogenous nucleic acid construct by at least one nucleotide. In some embodiments, the first recombination sequence of the first exogenous nucleic acid construct, the second recombination sequence of the first exogenous nucleic acid construct, the first recombination sequence of the second exogenous nucleic acid construct, and the second recombination sequence of the second exogenous nucleic acid construct each differ from each other by at least one nucleotide.

In a host cell provided herein, in some embodiments, the first recombination sequence is recognized by a tyrosine site-specific recombinase or a serine site specific recombinase. Optionally, the tyrosine site specific-recombinase is Cre recombinase or Flp recombinase. Optionally, the serine site specific recombinase is Bxb1 recombinase.

In some embodiments, a host provided herein is a mouse cell, a rat cell, a Chinese Hamster Ovary (CHO) cell, or a human cell.

In a host cell provided herein, in some embodiments, one or both of the nucleic acid constructs further comprise a promoter upstream of and a poly(A) sequence downstream of each of the respective genes encoding the antibody light chain, the antibody heavy chain, and the antibody heavy chain fusion protein.

In some embodiments, provided herein is a method for producing an antibody or antibody fusion protein, the method comprising: (a) providing the mammalian host cell as provided herein, and (b) culturing the host cell under conditions sufficient to express the antibody or antibody fusion protein. Optionally, the method further comprises recovering the expressed antibody or antibody fusion protein.

In some embodiments, provided herein is an antibody or antibody-fusion protein prepared by a host cell provided herein or according to the method provided herein.

In some embodiments, provided herein is a composition comprising a first expression vector and a second expression vector, wherein the first expression vector comprises in 5′ to 3′ order a first gene encoding an antibody light chain and a second gene encoding an antibody light chain, and wherein the second expression vector comprises in 5′ to 3′ order a first gene encoding an antibody heavy chain or antibody heavy chain fusion protein and a second gene encoding an antibody heavy chain.

In some embodiments, provided herein is a composition comprising a first expression vector and a second expression vector, wherein the first expression vector comprises the following elements in 5′ to 3′ order: a) first recombination sequence; b) first gene encoding an antibody light chain; c) second gene encoding an antibody light chain; d) second recombination sequence, and wherein the second expression vector comprises the following elements in 5′ to 3′ order: a) first recombination sequence; b) first gene encoding an antibody heavy chain or antibody heavy chain fusion protein; c) second gene encoding an antibody heavy chain; d) second recombination sequence.

In a composition provided herein, in some embodiments, the second expression vector first gene encodes an antibody heavy chain. In some embodiments, the second expression vector first gene encodes an antibody heavy chain fusion protein. In some embodiments, the first recombination sequence of the first expression vector has a different nucleotide sequence than the second recombination sequence of the first expression vector by at least one nucleotide. In some embodiments, the first recombination sequence of the first expression vector and the first recombination sequence of the second expression vector are recognized by the same recombinase enzyme. In some embodiments, the first recombination sequence of the first expression vector, the second recombination sequence of the first expression vector, the first recombination sequence of the second expression vector, and the second recombination sequence of the second expression vector are recognized by the same recombinase enzyme.

In a composition provided herein, in some embodiments, the first recombination sequence of the first expression vector has the same nucleotide sequence as the first recombination sequence of the second expression vector. In some embodiments, the first recombination sequence of the first expression vector has a different nucleotide sequence than the first recombination sequence of the second expression vector by at least one nucleotide. In some embodiments, the first recombination sequence of the first expression vector, the second recombination sequence of the first expression vector, the first recombination sequence of the second expression vector, and the second recombination sequence of the second expression vector each differ from each other by at least one nucleotide.

In a composition provided herein, in some embodiments, the first recombination sequence is recognized by a tyrosine site-specific recombinase or a serine site specific recombinase. Optionally, the tyrosine site specific-recombinase is Cre recombinase or Flp recombinase. Optionally, the serine site specific recombinase is Bxb1 recombinase.

In a composition provided herein, in some embodiments, one or both of the expression vectors further comprise a promoter upstream of and a poly(A) sequence downstream of each of the respective genes encoding the antibody light chain, the antibody heavy chain, and the antibody heavy chain fusion protein.

### BRIEF DESCRIPTION OF THE FIGURES/DRAWINGS

FIG. 1 is a schematic depicting the HC or LC transgenic ORF (rectangle) showing the NP location near the 5′ end of the ORF (vertical line in rectangle) and the corresponding primer/probe set (black lines above rectangle) used to detect each specific NP. The total primer probe set (dashed lines above rectangle) is located near the 3′ end of the ORF and is used to measure total transcript level.

FIG. 2 is a schematic depicting vector topologies integrated into each landing pad for the six different vector configurations (C1-C6). Light chain (LC) and heavy chain (HC) nucleotide polymorphisms (NP) are labeled NP1-NP4. Each NP remains consistent for each position in the vector and landing pad across all configurations. LC and HC ORFs and selective markers (“Select 1”-blasticidin resistance gene or “Select 2”-glutamine synthetase gene) are contained between the recombination sequences (“Rec”) of the vector to allow for proper integration into the host genome.

FIGS. 3A-3F depict bar graphs summarizing transcript expression results measuring cDNA levels for each mAb gene (identified by nucleotide polymorphism (NP)) in each vector for the 6 configurations outlined in FIG. 2, specifically configuration C1: FIG. 3A; configuration C2: FIG. 3B; configuration C3: FIG. 3C; configuration C4: FIG. 3D; configuration C5: FIG. 3E; configuration C6: FIG. 3F. For each of FIGS. 3A-3F, the bars contain data expression data for genes in order from left to right: Landing Pad A, position 1 (solid fill bar); Landing Pad A, position 2 (forward stripe bar); Landing Pad B, position 1 (checker fill bar); Landing Pad B, position 2 (reverse stripe bar). Results represent an average of triplicate ddPCR reactions from each of 2 or 3 replicate pools for every transfection condition.

FIG. 4A depicts a bar graph showing heavy and light chain cDNA transcripts quantified as a ratio against housekeeping gene GAPDH for both vector configuration C6 (LH LH) and vector configuration C2 (LL HH) as shown in FIG. 2 for six different monoclonal antibodies (mAb1-mAb6) compared to the theoretical (predicted) transcript levels for each configuration as predicted by the numerical key in Table 3. In FIG. 4A, for each mAb, the specific bars show: total normalized heavy chain (HC) transcripts in the C6 configuration (forward slash fill), total normalized HC transcripts in the C2 configuration (solid fill), total normalized light chain (LC) transcripts in the C6 configuration (empty fill), and total normalized LC transcripts in the C2 configuration (horizontal line fill). FIG. 4B shows the antibody titer results for each mAb in either the C6 vector configuration (empty fill) or the C2 vector configuration (solid fill) expressed as mg/L from a 12-day fed-batch for five different antibodies (mAb 4 titer data unavailable).

FIG. 5A depicts a bar graph showing predicted versus observed transcript levels for the vector configurations C1 and C2 for the mAb-fusion protein (see Table 4 for details of these configurations). From left to right, each group of 3 bars is for: i) configuration C1, predicted; ii) configuration C1, observed; iii) configuration C2, predicted; iv) configuration C2, observed. For each set of 3 bars, the specific bars show from left to right: total normalized heavy chain (HC) transcripts (solid fill); total normalized heavy chain-fusion protein (HC-F) transcripts (forward slash fill); and total normalized light chain (LC) transcripts (empty fill). FIG. 5B depicts a bar graph showing antibody titer results expressed as mg/L from a 12-day fed-batch for each vector configurations C1 and C2 for the mAb-fusion protein. FIG. 5C depicts a bar graph showing percent of each individual polypeptide chain for each of the C1 and C2 vector configurations for the mAb-fusion protein as determined by densitometry analysis of reduced SDS-PAGE. From left to right, each group of 3 bars is for: i) configuration C1; ii) configuration C2. For each set of 3 bars, the specific bars show from left to right: percent heavy chain (HC)(solid fill); percent heavy chain-fusion protein (HC-F)(forward slash fill); and percent light chain (LC) (empty fill)

### DETAILED DESCRIPTION

Disclosed herein are improved protein expression vectors, cells containing the vectors, and related compositions and methods.

The disclosure provided herein relates to the finding that certain configurations of antibody-encoding genes in protein expression vectors results in improved transcription of the genes, and of improved antibody product yield and/or quality.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995), as well as in subsequent editions and corresponding websites of the above references, as applicable.

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The following terms, unless otherwise indicated, shall be understood to have the following meanings:

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)₂, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy-chain constant regions that correspond to the different classes of immunoglobulins are called alpha, delta, epsilon, gamma, and mu, respectively. The subunit structures and three-dimensional configurations of different classes of immunoglobulins are well known.

The terms “polypeptide”, “oligopeptide”, “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

As known in the art, “polynucleotide,” or “nucleic acid,” as used interchangeably herein, refer to chains of nucleotides of any length and conformation (e.g. linear or circular) and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, a “recombinant” nucleic acid refers to a nucleic acid molecule that contains a polynucleotide sequence that does not occur in nature and/or or which is synthetically manufactured. For example, a “recombinant” nucleic acid may contain a protein-encoding gene coupled to a vector sequence. The sequence of the protein-encoding gene may occur in nature, but the gene does not naturally occur in combination with the vector sequence. Put another way, a “recombinant” nucleic acid molecule may contain as part of the molecule a nucleic acid sequence that occurs in nature, but that sequence is either coupled to another sequence (such that the totality of the nucleic acid molecule sequence does not occur in nature) and/or the molecule is synthetically manufactured. A “recombinant” polypeptide refers to a polypeptide produced from a recombinant nucleic acid.

As used herein, an “exogenous” nucleic acid refers to a recombinant nucleic acid molecule that will be or has been introduced into a host cell (e.g. by conventional genetic engineering methods, preferably by means of transformation, electroporation, lipofection, or transfection), which was prior to said introduction was not present in said host cell. In some circumstances, an exogenous nucleic acid contains a nucleotide sequence that does not naturally occur in the host cell. Such sequences are also termed “transgenic”. In some circumstances, an exogenous nucleic acid may contain a nucleotide sequence of that is the same as a sequence that is endogenous to the cell (i.e. an exogenous nucleic acid molecule may contain a nucleotide sequence of a gene that is endogenous to the host cell, such that introduction of the exogenous nucleic acid molecule into the host cell introduces an additional copy of the gene into the host cell). An “exogenous nucleic acid” refers to an exogenous nucleic acid molecule, or the nucleotide sequence thereof.

As used herein, a “nucleotide sequence of interest” refers to any nucleotide sequence that a person may want to introduce into a host cell or have present in a vector. A nucleotide sequence of interest may be in an exogenous nucleic acid. Most commonly, a nucleotide sequence of interest is a DNA sequence that encodes a polypeptide of interest or that is a template for the generation of an RNA molecule of interest. However, a nucleotide sequence of interest may alternatively, for example, be a sequence which provides a regulatory or structural function (e.g. a promoter or enhancer sequence), or which serves a different purpose, such as a restriction enzyme sequence for cloning purposes (e.g. a nucleotide sequence of interest may be a multiple cloning site). A nucleotide sequence of interest may be of any nucleotide length. A nucleotide sequence of interest may be a DNA sequence or an RNA sequence. In some embodiments, a nucleotide sequence of interest is a sequence that is not endogenously present in the host cell. In some embodiments, a nucleotide sequence of interest is separately endogenously present in the host cell (i.e. the sequence is also present in the host cell separate from a recombinant nucleic acid construct containing the nucleotide sequence of interest introduced into the host cell). In such embodiments, the nucleotide sequence of interest may be introduced into a host cell, for example, if there is relatively low expression of the corresponding endogenous nucleotide sequence, and it is desirable to have increased expression of the nucleotide sequence in the cell.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting. The term “or” when used in the context of a listing of multiple options (e.g. “A, B, or C”) shall be interpreted to include any one or more of the options, unless the context clearly dictates otherwise. It is understood that wherever embodiments are described herein with the language “comprising,” otherwise analogous embodiments described in terms of “consisting of” and/or “consisting essentially of” are also provided.

Exemplary methods and materials are described herein, although methods and materials similar or equivalent to those described herein can also be used in the practice or testing of the present invention. The materials, methods, and examples are illustrative only and not intended to be limiting.

### Vectors Containing Antibody-Encoding Genes

In one aspect, provided herein are vectors containing genes encoding antibody polypeptides. Genes encoding antibody polypeptides are also referred to as “antibody-encoding genes” herein.

As used herein, “antibody polypeptides” refer to antibody light chains, antibody heavy chains, antibody light chain fusion proteins, and antibody heavy chain fusion proteins. The terms “antibody heavy chain” and “antibody light chain” have the standard meaning in the art and include, for example, the various antibody heavy and light chains described elsewhere herein (e.g. heavy and light chains of IgG1, IgG2, IgG3, and IgG4 mAbs). The terms “antibody heavy chain” and “antibody light chain” include both standard full-length antibody heavy chains and light chains, as well as derivatives thereof that contain at the respective variable region (VL or VH). The term “antibody heavy chain fusion protein” refers to a polypeptide that contains an antibody heavy chain covalently linked to one or more additional proteins or peptides. For example, an “antibody heavy chain fusion protein” can be an antibody heavy chain covalently linked to a cytokine. The linkage may be direct, or via a peptide linker (e.g. a glycine-serine linker). In an antibody heavy chain fusion protein, the antibody heavy chain may be linked to additional protein(s) at the N-terminus or the C-terminus of the heavy chain (or both locations). The term “antibody light chain fusion protein” has the same meaning as described immediately above for “antibody heavy chain fusion protein”, except for an antibody light chain. As used herein, an “antibody fusion protein” refers to an antibody as provided herein which is covalently linked to one or more additional proteins or polypeptides (e.g. via a heavy chain or light chain of the antibody). Thus, an antibody fusion protein contains at least an antibody heavy chain fusion protein or an antibody light chain fusion protein as one of the polypeptides of the antibody fusion protein. Most commonly, an antibody fusion protein is a molecule that contains two antibody light chains, one antibody heavy chain, and one antibody heavy chain fusion protein, such that the additional protein is linked to one of the heavy chains of the antibody.

As used herein, “vector” means a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains 2 or more antibody-encoding genes, as well as one or more regulatory elements and/or selectable markers. For example, a vector as used herein may have the structure as described in Inniss, M, et al, Biotechnology and Bioengineering, 114(8): 1837-1846, 14 Mar. 2017, which is hereby incorporated by reference for all purposes. Vector components may include, for example, one or more of the following: a signal sequence; an origin of replication; one or more marker genes; suitable transcriptional controlling elements (such as promoters, enhancers and terminator). For expression (i.e., translation), one or more translational controlling elements may also be included such as ribosome binding sites, translation initiation sites, and stop codons. A vector may contain a “nucleic acid construct”.

A “nucleic acid construct” as provided herein is a type of polynucleotide or nucleic acid described herein. A “nucleic acid construct” may have any of the characteristics of a polynucleotide or nucleic acid described herein. Typically, a “nucleic acid construct” as provided herein contains two or more functional units within the chain of nucleotides that make up the polynucleotide. A functional unit in a nucleotide sequence may be any type of discrete nucleotide sequence having a particular function such as, for example, a nucleotide sequence of interest, a gene encoding a polypeptide, a regulatory sequence, or a recombination sequence. In the context of a vector, a “nucleic acid construct” may have the features of an “expression cassette” (also referred to herein simply as a “cassette”) within a larger vector sequence. For example, a nucleic acid construct may include an antibody encoding gene and associated regulatory elements. Specifically, a nucleic acid construct may include, for example at least i) a promoter sequence; ii) an antibody-encoding gene; and iii) a 3′ untranslated region. In some embodiments, a nucleic acid construct may have the features of a “cassette” as used in recombinase mediated cassette exchange (RMCE). (See, e.g. Inniss, M, et al, Biotechnology and Bioengineering, 114(8): 1837-1846, 14 Mar. 2017; Zhang L et al, Biotechnol Prog 31(6): 1645-1656, 2015; Turan, S et al, Gene, 515(1): 1-27, 2013; Crawford, Y et al, Biotechnol Prog, 29(5): 1307-1315, 2013; Kawabe Y et al, Cytotechnology, 64(3): 267-279, 2012; and Kim M S and Gyun M L, J Microbiol Biotechnol 18(7): 1342-1351, 2008; which are hereby incorporated by references for all purposes). For example, a “nucleic acid construct” may have one or more features of vector components as described in Inniss, M, et al, Biotechnology and Bioengineering, 114(8): 1837-1846, 14 Mar. 2017, which is hereby incorporated by reference for all purposes. For example, as depicted in Inniss page 1838, right column, an “RMCE mAb targeting vector” contains the following elements in sequence (between the FRT F5 Bxb1 attB site and the Bxb1 mut attB FRT wt site): a) selectable marker (blasticidin gene); b) SV40 polyA sequence; c) human CMV (hCMV) promoter; d) antibody-encoding gene (light chain); e) SV40 polyA sequence; f) hCMV promoter; g) antibody-encoding gene (heavy chain); h) SV40 polyA sequence. Thus, as used herein, a “nucleic acid construct” may contain, for example, some or all of these elements.

Exemplary promoters include the SV40 early promoter and the human CMV promoter.

Exemplary selectable markers include antibiotic resistance genes for blasticidin, puromycin, or geneticin (G418).

Typically, a vector or nucleic acid construct provided herein contains at least two antibody-encoding genes. The two antibody-encoding genes may be the same or different genes. For example, in some embodiments, a vector or nucleic acid construct may contain two copies of the same antibody light chain gene. Alternatively, in some embodiments, a vector or nucleic acid construct may contain one copy of an antibody light chain gene and one copy of an antibody heavy chain gene, or one copy of an antibody light chain gene from a first antibody and one copy of an antibody light chain gene from a second antibody. In a vector containing a first antibody gene and a second antibody gene, the upstream (i.e. first transcribed in the 5′ to 3′ direction) position is referred to herein as the “first position”, and the downstream position is referred to herein as the “second position”.

In some embodiments provided herein, at least two vectors are used. Both vectors are introduced into the same host cell. Accordingly, also provided herein are related compositions, methods, and host cells containing two vectors.

When at least two vectors are used in embodiments provided here, the at least two vectors may have at least two different configurations. For example, in an embodiment having two configurations, there is a “first vector” configuration and a “second vector” configuration. A first vector is considered as having different configuration from a second vector if at least one element or position of an element differs between the first and second vector. As used herein, a first vector and a second vector having different configurations may be referred to as a “first vector” and “second vector”

In embodiments provided herein involving a first vector and a second vector, in some aspects, the first vector contains a first gene encoding an antibody light chain and a second gene encoding an antibody light chain, and the second vector contains a first gene encoding an antibody heavy chain and a second gene encoding an antibody heavy chain. In some embodiments, the first gene encoding the antibody light chain and the second gene encoding the antibody light chain encode the same antibody light chain (e.g. two copies of the same gene). In some other embodiments, the first gene encoding the antibody light chain and the second gene encoding the antibody light chain encode different antibody light chains (e.g. one copy each of two different antibody light chain genes). In some embodiments, the first gene encoding the antibody heavy chain and the second gene encoding the antibody heavy chain encode the same antibody heavy chain (e.g. two copies of the same gene). In some other embodiments, the first gene encoding the antibody heavy chain and the second gene encoding the antibody heavy chain encode different antibody heavy chains (e.g. one copy each of two different antibody heavy chain genes).

In one example, for expressing standard IgG monospecific antibodies, it may be desirable to use embodiments in which the first gene encoding the antibody light chain and the second gene encoding the antibody light chain encode the same antibody light chain, and the first gene encoding the antibody heavy chain and the second gene encoding the antibody heavy chain encode the same antibody heavy chain. This is useful because a standard IgG monospecific antibody contains only two different polypeptide types: i) antibody light chain and ii) antibody heavy chain. Accordingly, for standard IgG expression it is possible to have both the first and second position in the first vector contain the same gene encoding the same antibody light chain, and to have both the first and second position in the second vector contain the same gene encoding the same antibody heavy chain.

In another example, for expressing bispecific IgG antibodies, it may be desirable to use embodiments in which the first gene encoding the antibody light chain and the second gene encoding the antibody light chain encode different antibody light chains, and the first gene encoding the antibody heavy chain and the second gene encoding the antibody heavy chain encode the different antibody heavy chains. This is useful because typical bispecific IgG antibodies contain four different polypeptide types: i) antibody light chain for the first antigen binding portion, ii) antibody heavy chain for the first antigen binding portion, iii) antibody light chain for the second antigen binding portion, and iv) antibody heavy chain for the second antigen binding portion. Accordingly, for bispecific IgG expression it may be desirable to have the first position in the first vector containing a gene encoding a first antibody light chain, the second position in the first vector containing a gene encoding a second antibody light chain, the first position in the second vector containing a gene encoding a first antibody heavy chain, and the second position in the second vector containing a gene encoding a second antibody heavy chain.

Other related embodiments are also provided herein. For example, in some embodiments a first vector and second vector may be used with embodiments provided here, such as for the expression of a bispecific antibody having a common light chain (see, e.g. WO2021/124073, published 24 Jun. 2021). A bispecific antibody having a common light chain may contain two different heavy chains (e.g. having In this circumstance, optionally, the first and second position in the first vector contain the same gene encoding the same antibody light chain, and the first position in the second vector contains a gene encoding a first antibody heavy chain, and the second position in the second vector contains a gene encoding a second antibody heavy chain.

In embodiments provided herein involving a first vector and a second vector, in some other aspects, the first vector contains a first gene encoding an antibody light chain and a second gene encoding an antibody light chain, and the second vector contains a first gene encoding an antibody heavy chain fusion protein and a second gene encoding an antibody heavy chain. In some embodiments, the first gene encoding the antibody light chain and the second gene encoding the antibody light chain encode the same antibody light chain (e.g. two copies of the same gene). In some other embodiments, the first gene encoding the antibody light chain and the second gene encoding the antibody light chain encode different antibody light chains (e.g. one copy each of two different antibody light chain genes).

The choice of whether to have two copies of the same antibody light chain or one copy each of two different antibody light chains in the first vector depends on specific type of antibody molecule to be manufactured. For example, for an antibody fusion protein involving a standard monospecific IgG mAb covalently linked to another protein via the antibody heavy chain, the molecule typically contains 3 separate polypeptide types: i) antibody light chain; ii) antibody heavy chain; and iii) antibody heavy chain fusion protein. In this antibody fusion protein molecule there is one chain each of the antibody heavy chain and antibody heavy chain fusion protein, and two copies of the antibody light chain. Since there is only one type of antibody light chain in this molecule, two copies of the same antibody light chain gene are provided in the first vector. In another example, for an antibody fusion protein involving a bispecific IgG mAb covalently linked to another protein via the antibody heavy chain, the molecule typically contains 4 separate polypeptide types: i) antibody light chain for the first antigen binding portion; ii) antibody heavy chain for the first antigen binding portion; iii) antibody light chain for the second antigen binding portion; and iv) antibody heavy chain fusion protein, in which the antibody heavy chain portion is for the second antigen binding portion. In this antibody fusion protein molecule there is one copy each of the antibody heavy chain and antibody heavy chain fusion protein, and one copy of each of the antibody light chains. Since there are two types of antibody light chain in this molecule, two different antibody light chain genes are provided in the first vector.

In embodiments provided herein involving two different antibody heavy chains or an antibody heavy chain and an antibody heavy chain fusion protein, optionally, the heavy chains may contain one or more amino acid modifications in the constant region of the heavy chains to promote heterodimer formation between the two different heavy chains. Such modifications are known in the art and include, for example, charged-based electrostatic amino acid modifications, and steric-based “knob into hole” amino acid modifications.

In some embodiments, a nucleic acid construct as provided herein is flanked on one or both ends of the construct with a recombination sequence. A “recombination sequence” or a “recombination site” is a stretch of nucleotides being necessary for and allowing, together with a recombinase, a targeted recombination and defining the location of such a recombination. As used herein, “recombination sequence” is typically used to refer to a recombination sequence on an exogenous nucleic acid construct to be introduced into a host cell, and “recombination site” is typically used to refer to a corresponding recombination sequence in a host cell chromosome. A recombination site may be non-native to a host cell genome (e.g. it may be introduced into a host cell chromosome as part of a landing pad sequence).

In some embodiments, one or more recombination sequences may be included in a nucleic acid construct provided herein, so that some or all of the nucleic acid construct may be integrated into a corresponding site at in a host cell chromosome.

Any suitable recombination site, target sequence and recombinase combination may be used with the compositions and methods provided herein, including both tyrosine recombinase and serine recombinase-based systems. Recombinases (and their corresponding recombination sequences) that may be used with nucleic acid constructs and host cells provided herein include, for example, Cre, Dre, Flp, KD, B2, B3, λ, HK022, HP1, γδ, ParA, Tn3, Gin, Bxb1, φC31, φBT1, and R4. Site specific recombinases are described, for example, in Turan and Bode, The FASEB Journal, 25 (12): 4088-107 (2011); Nern et al, PNAS, 108 (34): 14198-203 (2011); and Xu et al, BMC Biotechnology, 13 (87) (2013).

As noted above, in some embodiments, a nucleic acid construct provided herein may be or have the characteristics of a “cassette” as used in RMCE. In these embodiments, a nucleic acid construct is flanked on one or both ends of the construct with a recombination sequence. In embodiments in which the nucleic acid construct is flanked at both ends with recombination sequences, in some embodiments, the recombination sequences on either end have the same sequence. In other embodiments, the recombination sequences on either end have different sequences. It may be desirable to provide different recombination sequences on either end of a nucleic acid construct, in order to permit directional insertion of a nucleic acid construct/cassette in a landing pad in a host cell (discussed further below). For example, various different recombination sequences are orthogonal to each other (i.e. they do not cross-combine) and may be used to prepare nucleic acid constructs that can be directionally inserted into a landing pad in a host cell. For example, the Bxb1 wild-type and Bxb1-GA mutant sequences are orthogonal (Jusiak, B, et. al, AC Synth. Biol., 2019, 8(1), pp 16-24), and can be used on either end of a nucleic acid construct for directional insertion via Bxb1 recombinase. In another example, wild-type FRT and FRT F5 mutant sequence can be used in either end of a nucleic acid construct for directional insertion via Flp recombinase.

In some embodiments, a first vector provided herein contains a first nucleic acid construct, wherein the first nucleic acid construct is flanked on the 5′ end by a first recombination sequence and on the 3′ end by a second recombination sequence, and a second vector provided herein contains a second nucleic acid construct, wherein the second nucleic acid construct is flanked on the 5′ end by a first recombination sequence and on the 3′ end by a second recombination sequence. Optionally, the first recombination sequence of the first vector has a different nucleotide sequence than the second recombination sequence of the first vector. Optionally, the first recombination sequence of the first vector has the same nucleotide sequence as the second recombination sequence of the first vector. Optionally, the first recombination sequence of the second vector has a different nucleotide sequence than the second recombination sequence of the second vector. Optionally, the first recombination sequence of the second vector has the same nucleotide sequence as the second recombination sequence of the section vector. Optionally, each of i) the first recombination sequence of the first vector, ii) the second recombination sequence of the first vector, iii) the first recombination sequence of the second vector, and iv) the second recombination sequence of the second vector has a different nucleotide sequence.

In some embodiments, an antibody-encoding gene in a nucleic acid construct may be linked to one or more regulatory genetic control elements in the nucleic acid construct. In certain embodiments, a genetic control element directs constitutive expression of the nucleotide sequence of interest. In certain embodiments, a genetic control element that provides inducible expression of a nucleotide sequence of interest can be used. The use of an inducible genetic control element (e.g., an inducible promoter) allows for modulation of the production of, for example, a polypeptide encoded by a gene. Non-limiting examples of potentially useful inducible genetic control elements for use in eukaryotic cells include hormone-regulated elements (e.g., see Mader, S. and White, J. H., Proc. Natl. Acad. Sci. USA 90:5603-5607, 1993), synthetic ligand-regulated elements (see, e.g. Spencer, D. M. et al., Science 262:1019-1024, 1993) and ionizing radiation-regulated elements (e.g., see Manome, Y. et al., Biochemistry 32:10607-10613, 1993; Datta, R. et al., Proc. Natl. Acad. Sci. USA 89:10149-10153, 1992). Additional cell-specific or other regulatory systems known in the art may be used in accordance with the methods and compositions provided herein.

Polynucleotides provided herein may be single-stranded (coding or antisense) or double-stranded, and may be DNA (genomic, cDNA or synthetic) or RNA molecules. Additional coding or non-coding sequences may, but need not, be present within a polynucleotide of the present invention, and a polynucleotide may, but need not, be linked to other molecules and/or support materials. Polynucleotides complementary to any nucleic acid construct or vector sequences provided herein are also encompassed by the present invention. It will be appreciated by those of ordinary skill in the art that, as a result of the degeneracy of the genetic code, there may be multiple nucleotide sequences that encode a polypeptide provided herein.

Polynucleotides provided herein can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g. without limitation, pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

### Host Cells

As used herein, the term “host cell”, refers to a cell or cell culture harboring a recombinant nucleic acid provided herein, or that can be a recipient for such nucleic acids. Host cells include progeny of a single host cell.

In some embodiments, a host cell may harbor a recombinant nucleic acid stably integrated at a location in its genome (e.g. in a chromosome). In some embodiments, a recombinant nucleic acid in a host cell is not stably integrated into the host cell's genome—e.g. the recombinant nucleic acid may be in the host cell in a plasmid.

In the context of the present disclosure, a “cell” is preferably a mammalian cell. A mammalian cell may be, for example, a canine cell (e.g. Madin-Darby canine kidney epithelial (MDCK) cell), a primate cell, a human cell (e.g. human embryonic kidney (HEK) cell), a mouse cell or a hamster cell. In some embodiments, a hamster cell is a Chinese hamster ovary (CHO) cell. Optionally, a CHO cell may be a CHOK1, a CHOK1 SV cell (Porter, A J et al. Biotechnol Prog. 26 (2010), 1455-1464), or another strain of CHO cell. In some embodiments, a mammalian cell is a BALB/c mouse myeloma cell, a human retinoblast cell (PER.C6), a monkey kidney cell, a human embryonic kidney cell (293), a baby hamster kidney cell (BHK), a mouse sertoli cell, an African green monkey kidney cell (CERO-76), a HeLa cell, a buffalo rat liver cell, a human lung cell, a human liver cell, a mouse mammary tumor cell, a TRI cell, a MRC 5 cell, a FS4 cell, or a human hepatoma cell (e.g. Hep G2). In some embodiments, a cell is a non-mammalian cell (e.g. an insect cell or a yeast cell).

Introduction of Polynucleotides into Cells

Polynucleotides provided herein (e.g. nucleic acid constructs, vectors, etc.) can be introduced into a host cell by any of a number of appropriate means, including, for example, electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of method for introduction of a polynucleotide into a host cell will often depend on features of the host cell.

Methods suitable for introducing nucleic acids sufficient to achieve expression of a protein of interest into mammalian host cells are known in the art. See, for example, Gething et al., Nature, 293:620-625, 1981; Mantei et al., Nature, 281:40-46, 1979; Levinson et al. EP 117,060; and EP 117,058, each of which is incorporated herein by reference. For mammalian cells, common methods of introducing genetic material into mammalian cells include the calcium phosphate precipitation method of Graham and van der Erb (Virology, 52:456-457, 1978) or the Lipofectamine™ (Gibco BRL) method of Hawley-Nelson (Focus 15:73, 1993). General aspects of mammalian cell host system transformations have been described by Axel in U.S. Pat. No. 4,399,216 issued Aug. 16, 1983. For various techniques for introducing genetic material into mammalian cells, see Keown et al., Methods in Enzymology, 1989, Keown et al., Methods in Enzymology, 185:527-537, 1990, and Mansour et al., Nature, 336:348-352, 1988. Additional methods suitable for introducing nucleic acids include electroporation, for example as employed using the GenePulser XCell™ electroporator by BioRad™ or Neon Electroporation by ThermoFisher. Non-limiting representative examples of suitable vectors for expression of proteins in mammalian cells include pCDNA1; pCD, see Okayama, et al. Mol. Cell Biol. 5:1136-1142, 1985; pMCIneo Poly-A, see Thomas, et al. Cell 51:503-512, 1987; a baculovirus vector such as pAC 373 or pAC 610; CDM8, see Seed, B. Nature 329:840, 1987; and pMT2PC, see Kaufman, et al. EMBO J. 6:187-195, 1987, each of which is incorporated herein by reference in its entirety.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther., 1992, 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther., 1992, 3:147); ligand-linked DNA (see, e.g., Wu, J. Biol. Chem., 1989, 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes.

Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP 0524968. Additional approaches are described in Philip, Mol. Cell Biol., 1994, 14:2411, and in Woffendin, Proc. Natl. Acad. Sci., 1994, 91:1581. Naked DNA can be introduced into cells by forming a precipitate containing the DNA and calcium phosphate. Alternatively, naked DNA can also be introduced into cells by forming a mixture of the DNA and DEAE-dextran and incubating the mixture with the cells or by incubating the cells and the DNA together in an appropriate buffer and subjecting the cells to a high-voltage electric pulse (e.g., by electroporation). Naked DNA can also be directly injected into cells by, for example, microinjection. Alternatively, naked DNA can also be introduced into cells by complexing the DNA to a cation, such as polylysine, which is coupled to a ligand for a cell-surface receptor (see for example Wu, G. and Wu, C. H. J. Biol. Chem. 263:14621, 1988; Wilson et al. J. Biol. Chem. 267:963-967, 1992; and U.S. Pat. No. 5,166,320, each of which is hereby incorporated by reference in its entirety). Binding of the DNA-ligand complex to the receptor facilitates uptake of the DNA by receptor-mediated endocytosis.

In certain embodiments, a polynucleotide provided herein is stably introduced into a host cell. In certain embodiments, a polynucleotide provided herein is transiently introduced into the host cell.

Integration of Nucleic Acids into Host Cell Chromosomes

In embodiments provided herein in which a polynucleotide is stably introduced into a host cell (for example, in situations where the polynucleotide is integrated into a host cell chromosome), the polynucleotide may be randomly integrated into a chromosome in the host cell, or the polynucleotide may be integrated at a specific location in a chromosome in the host cell. These approaches may be referred herein to as a “random integration” or “site-specific integration (“SSI”)”, respectively.

For random integration, typically, one or more recombinant nucleic acid constructs are prepared in which the recombinant nucleic acid construct(s) each contain at least one nucleotide sequence of interest and at least one selectable marker (e.g. a gene encoding antibiotic resistance).

After preparation of the polynucleotide(s) containing one or more of the antibody-encoding genes, the polynucleotides can be introduced in to host cells. Host cells that have taken up the polynucleotides can be selected, for example, by resistance to the antibiotic for which the antibiotic resistance gene in the construct provides resistance. Generally, after polynucleotide(s) containing the genes of interest are introduced into a population of cells, and cells are selected for via the relevant selectable marker system (e.g. antibiotic resistance) there may be a heterogeneous population of cells (also referred to herein as a “pool” of cells) containing different numbers of copies of the polynucleotide(s) containing the one or more antibody-encoding genes, as well as different locations of integration of the polynucleotide(s) in chromosomes in the cell. Optionally, individual cells from this pool of cells may be sorted and isolated, and individual homogenous cell line populations of different cells may be established (also referred to herein as cell line “clones”). Alternatively, in some embodiments, a heterogeneous pool of cells containing a first and second nucleic acid construct as provided herein genes may be maintained. Either type of cell population described above (e.g. homogenous or heterogeneous populations) may be used for various methods (e.g. protein production) as described herein.

In some embodiments, nucleic acid constructs for random integration may be linear polynucleotides. In some embodiments, the linear structure may be generated by synthesis of a linear molecule (e.g. by PCR or chemical polynucleotide synthesis). In some embodiments, the linear structure may be generated by cleavage of a circular vector (e.g. by a restriction enzyme) to generate a linear nucleic acid molecule.

In some embodiments, provided herein is a host cell comprising one or more nucleic acid constructs provided herein integrated into a chromosome of the cell. For example, in some embodiments, provided herein is a host cell comprising a first recombinant nucleic acid construct and a second recombinant nucleic acid construct provided herein integrated into a chromosome of the cell.

For site-specific integration, in some embodiments, a host cell that contains one or more “landing pads” at one or more defined chromosomal locus is used. The landing pad contains an exogenous nucleotide sequence that contains one or more recombination sites, which is stably integrated into a chromosome. When an exogenous nucleic acid construct that contains one or more recombination sequences that correspond to the recombination site in the landing pad is introduced into the host cell, an expression cassette in the exogenous nucleic acid construct may be integrated into or replace the landing pad sequence (for example, via recombinase mediated cassette exchange (RMCE)). In some embodiments, an SSI system as described, for example, in Zhang L, et. al (Biotechnol Prog. 2015; 31: 1645-1656) or International Publication WO 2013/190032 may be used with embodiments provided herein, which are hereby incorporated by reference for all purposes.

In some embodiments, a host cell for use with compositions and methods provided here contains at least two separate landing pads. Optionally, a first vector or first nucleic acid construct as provided herein may contain recombination sequences that correspond to the recombination site in the first landing pad, and a second vector or second nucleic acid construct as provided herein may contain recombination sequences that correspond to the recombination site in the second landing pad in the cell. In some embodiments, multiple landing pad sites in a CHO cell may be used as described in US2020/0002727, published Jan. 2, 2020. In some embodiments, a landing pad may be located in the CHO Fer1L4 gene, as described in WO2013/190032, published Dec. 27, 2013. In some embodiments, a landing pad may be located in the NL1 locus as described in US2020/0002727, published Jan. 2, 2020.

In some embodiments, a landing pad in a host cell line may be located at a “hot-spot” in the host cell's genome. As used herein, the term “hot-spot” means a site, in the genome of a host cell which provides for a stable and high expression of a gene or genes integrated at the site.

A cell that contains a landing pad for SSI may also be referred to herein as a “SSI host cell”. As used herein, “SSI host cell” refers to a host cell that contains an exogenous nucleotide sequence that includes at least one recombination site (e.g. a landing pad). The recombination site in the host cell permits site specific integration of exogenous nucleotide sequences into the genome of the host cell, thus enabling a predetermined localized and directed integration of desired nucleotide sequences at a desired place in a host cell's genome. Thus, in some embodiments, a site specific integration host cell is capable of targeted integration of a recombinant nucleic acid construct (or an expression cassette therein) described herein into a chromosome of the host cell. In some embodiments, a site specific integration host cell is capable of targeted integration of an expression cassette by recombination mediated cassette exchange (RMCE).

For compositions and methods provided herein involving recombination of an exogenous nucleic acid construct into a host cell genome, as described above, a recombinase is also present or introduced into the host cell. Methods provided herein involving introducing an exogenous nucleic acid construct may include introducing a gene encoding a recombinase into the host cell.

In some embodiments, provided herein is a host cell comprising an exogenous recombinant nucleic acid construct integrated into a specific location in a chromosome in the cell. The nucleic acid construct may have any of the properties of a nucleic acid construct provided herein.

### Recombinant Antibodies

In another aspect, provided herein are recombinant antibodies (also referred to herein as “recombinant proteins” and “recombinant polypeptides”) that are produced via the compositions and methods provided herein. For example, provided herein is an antibody that is encoded by an antibody-encoding gene that is a component of a recombinant nucleic acid construct provided herein.

Any antibody that is expressible in a host cell may be produced in accordance with the present teachings and may be produced according to the methods of the invention or by the cells of the invention.

### Isolation of the Expressed Antibody

In general, it will typically be desirable to isolate and/or purify antibodies expressed according to the present invention. In certain embodiments, the expressed antibody is secreted into the medium and thus cells and other solids may be removed, as by centrifugation or filtering for example, as a first step in the purification process. Alternatively, the expressed antibody may remain in the cell or may be bound to the surface of the host cell. In such circumstances, the media may be removed and the host cells expressing the protein are lysed as a first step in the purification process. Lysis of mammalian host cells can be achieved by any number of means well known to those of ordinary skill in the art, including physical disruption by glass beads and exposure to high pH conditions.

The expressed antibody may be isolated and purified by standard methods including, but not limited to, chromatography (e.g., ion exchange, affinity, size exclusion, and hydroxyapatite chromatography), gel filtration, centrifugation, or differential solubility, ethanol precipitation and/or by any other available technique for the purification of proteins (See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol. 182), Academic Press, 1997, each of which is incorporated herein by reference). For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the protein during the purification process. Protease inhibitors are particularly advantageous when cells must be lysed in order to isolate and purify the expressed antibody.

### Cell Cultures and Cell Culture Media

The terms “medium”, “media”, and the like as used herein refer to a solution containing components or nutrients which nourish growing mammalian cells. Typically, the nutrients include essential and non-essential amino acids, vitamins, energy sources, lipids, and trace elements required by the cell for minimal growth and/or survival. Such a solution may also contain further nutrients or supplementary components that enhance growth and/or survival above the minimal rate, including, but not limited to, hormones and/or other growth factors, particular ions (such as sodium, chloride, calcium, magnesium, and phosphate), buffers, vitamins, nucleosides or nucleotides, trace elements (inorganic compounds usually present at very low final concentrations), inorganic compounds present at high final concentrations, amino acids, lipids, and/or glucose or other energy source. In some embodiments, a medium is advantageously formulated to a pH and salt concentration optimal for cell survival and proliferation. In some embodiments, a medium is a feed medium that is added after the beginning of the cell culture.

A wide variety of mammalian growth media may be used in accordance with the present invention. In some embodiments, cells may be grown in one of a variety of chemically defined media, wherein the components of the media are both known and controlled. In some embodiments, cells may be grown in a complex medium, in which not all components of the medium are known and/or controlled.

Chemically defined growth media for mammalian cell culture have been extensively developed and published over the last several decades. All components of defined media are well characterized, and so defined media do not contain complex additives such as serum or hydrolysates. Early media formulations were developed to permit cell growth and maintenance of viability with little or no concern for protein production. More recently, media formulations have been developed with the express purpose of supporting highly productive recombinant protein producing cell cultures. Such media are preferred for use in the method of the invention. Such media generally comprises high amounts of nutrients and in particular of amino acids to support the growth and/or the maintenance of cells at high density. If necessary, these media can be modified by the skilled person for use in the method of the invention. For example, the skilled person may decrease the amount of phenylalanine, tyrosine, tryptophan and/or methionine in these media for their use as base media or feed media in a method as disclosed herein.

In some embodiments, methods and compositions provided herein involve cell cultures and cell culture media. The terms “culture” and “cell culture” as used herein refer to a cell population that is in a medium under conditions suitable to survival and/or growth of the cell population. As will be clear to those of ordinary skill in the art, in some embodiments, these terms as used herein refer to the combination comprising the cell population and the medium in which the population is present. In some embodiments, the cells of the cell culture comprise mammalian cells. In some embodiments, a cell culture comprises cells in suspension. In some embodiments, a cell culture comprises cells grown on a substrate.

In some embodiments, host cells provided herein which contain a recombinant nucleic acid construct provided herein may be used to produce an antibody encoded by a first nucleic acid construct and second nucleic acid construct provided herein. Similarly, as provided herein, methods and compositions provided herein may be used to obtain host cells that contain a first nucleic acid construct and second nucleic acid construct provided herein, and polypeptides encoded by such nucleic acid constructs may be produced and purified. In addition, such host cells may be generated and cultured.

The present invention may be used with any cell culture method that is amenable to the desired process (e.g., introduction of a recombinant nucleic acid construct according to methods provided herein and production of a recombinant protein (e.g., an antibody)). As a non-limiting example, cells may be grown in batch or fed-batch cultures, where the culture is terminated after sufficient expression of the recombinant protein (e.g., antibody), after which the expressed protein (e.g., antibody) is harvested. Alternatively, as another non-limiting example, cells may be grown in batch-refeed, where the culture is not terminated and new nutrients and other components are periodically or continuously added to the culture, during which the expressed recombinant protein (e.g., antibody) is harvested periodically or continuously. Other suitable methods (e.g., spin-tube cultures) are known in the art and can be used to practice the present invention.

In some embodiments, provided herein are compositions containing antibodies produced from host cells and according to methods provided herein, and one or more pharmaceutically acceptable carriers, excipients, or stabilizers (Remington: The Science and practice of Pharmacy 20th Ed., 2000, Lippincott Williams and Wilkins, Ed. K. E. Hoover), in the form of lyophilized formulations or aqueous solutions. Acceptable carriers, excipients, or stabilizers are nontoxic to recipients at the dosages and concentrations, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

### Methods

In some aspects, provided herein are methods of selecting host cells provided herein, methods for preparing host cells provided herein, and methods for producing recombinant antibodies provided herein.

In some embodiments, nucleic acid constructs or vectors containing such nucleic acid constructs as described herein may be prepared by standard techniques as known in the art for preparing recombinant nucleic acids.

Host cells containing nucleic acid constructs as provided herein may be prepared by introducing the relevant nucleic acid constructs into the host cells by methods known in the art, as described elsewhere herein.

In some embodiments, provided herein are methods for producing antibodies. According to such methods, cells provided herein (including cells selected according to methods provided herein or prepared according to methods provided herein) can be used, for example, for the expression of an antibody or antibody fusion protein encoded by a first nucleic acid construct and a second nucleic acid construct in a host cell.

One of ordinary skill in the art will appreciate that the exact purification technique will vary depending on the character of the antibody to be purified, the character of the cells from which the protein is expressed, and/or the composition of the medium in which the cells were grown.

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1: Assessment of Order and Positional Effects of mAb-Encoding Genes in Vectors in a Dual-Landing Pad CHO Cell Line

### Goal:

In this example, the effect of location in expression vectors on transcription and mAb titer of antibody heavy chain and light chain encoding genes in a dual-landing pad CHO cell line was assessed.

### Materials and Methods

### Modification of mAb Genes to Assess Order and Positional Effects

To assess the order and positional effects of antibody heavy chain gene and light chain gene location within expression vectors on mAb gene transcription, silent mutations were engineered into the heavy and light chain open reading frames of a monoclonal antibody (“mAb1”) for a single arginine near the 5′ end of the respective sequence. These mutations, or nucleotide polymorphisms (NPs), were used as biomarkers to track the level of transcript being generated from each expression vector position. Arginine was selected due to there being six different codons that can be used as NPs, and are represented in approximate equal frequency in the CHO genome. Four different NPs were engineered for each heavy chain gene (HC) and light chain gene (LC) in order to assess four different expression vector positions. The different NPs are shown below in Table 1.

TABLE 1
Nucleotide Polymorphisms

| Nucleotide | | |
|---|---|---|
| Polymorphism | Light Chain (LC) Codon | Heavy Chain (HC) Codon |
| NP1 | CGT | AGA |
| NP2 | AGG | AGG |
| NP3 | CGG | CGG |
| NP4 | CGA | CGA |

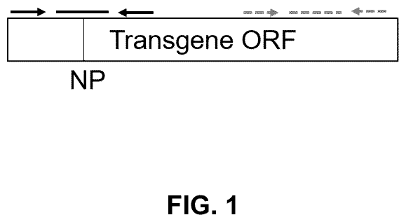

The nucleotide polymorphisms were quantified using ddPCR with probes utilizing locked nucleic acid technology against total heavy and light chain transcript levels, as measured by a separate primer/probe set near the 3′ end of the molecule (FIG. 1). Heavy chain genes, light chain genes, and other genes (e.g. GAPDH) are also referred to herein as “open reading frames” (ORF).

### Expression Vectors

Two different expression vectors were designed for use with a dual-landing pad host cell line, where each vector is targeted to one of the two independent landing pads in the host cell (“Landing Pad A” and “Landing Pad B”) and is integrated into a host-cell chromosome via recombinase-mediated insertion. The landing pads and vectors have a similar structure as described in Inniss, M, et al, Biotechnology and Bioengineering, 114(8): 1837-1846, 14 Mar. 2017, which is hereby incorporated by reference for all purposes. To ensure occupancy of each landing pad, one expression vector confers blasticidin resistance [blasticidin resistance gene; see Kimura M et al, Biochim Biophys Acta 1219(3): 653-659, 1994] the other enables glutamine synthesis in the otherwise glutamine synthetase knock out host (glutamine synthetase gene; see Matasci, M et al, Drug Discovery Today Technol. 5, e37-42, 2008). Each expression vector contains two multiple cloning sites for open reading frame insertion. Each open reading frame is flanked by a hCMV promoter and a SV40 polyA tail 3′ to the insertion site. These vectors allow for a total of at least four transgenes to be inserted into the host cell genome (2 transgenes per vector), each of which is flanked by the same regulatory sequences.

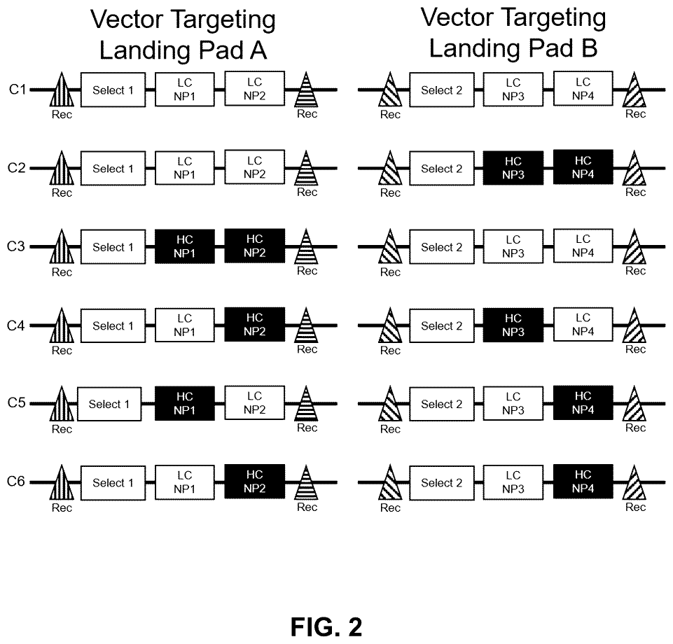

Expression vectors were generated to assess varying positions and ordering of heavy and light chain. Six different combinations of expression vector configuration were prepared having the configurations as shown in FIG. 2. (The schematic in FIG. 2 does not include all features in the vector, such as promoters and polyA tails.) The configuration of the antibody genes in the different vectors in FIG. 2 is summarized in Table 2.

TABLE 2
Expression Vector Configurations

| | Vector for | | Vector for | |
|---|---|---|---|---|
| | Landing Pad A | | Landing Pad B | |
| Configuration | Gene + NP | Gene + NP | Gene + NP | Gene + NP |
| # | Position 1 | Position 2 | Position 1 | Position 2 |
| C1 | LC NP1 | LC NP2 | LC NP3 | LC NP4 |
| C2 | LC NP1 | LC NP2 | HC NP3 | HC NP4 |
| C3 | HC NP1 | HC NP2 | LC NP3 | LC NP4 |
| C4 | LC NP1 | HC NP2 | HC NP3 | LC NP4 |
| C5 | HC NP1 | LC NP2 | LC NP3 | HC NP4 |
| C6 | LC NP1 | HC NP2 | LC NP3 | HC NP4 |

Expression vectors were constructed by amplifying the gene of interest by PCR using Q5® Hot Start High-Fidelity 2× Master Mix (New England BioLabs). During amplification, oligo tags were added to target genes of interest into the desired locations of the restriction digested expression vector using NEBuilder® HiFi DNA Assembly Master Mix (New England BioLabs). Nucleotide polymorphisms were introduced to the 5′ end of the open reading frame using oligos to incorporate silent mutations via overlap extension amplification.

### Host Cells

The host cell was an in-house Pfizer host CHO cell line (a CHOK1 derivative) that was genetically engineered to contain two independent landing pads (“Landing Pad A” and “Landing Pad B”) in areas of the genome that have been shown to be highly transcriptionally active.

### Transfection and Routine Cell Culture

Triplicate transfections were performed for each expression vector configuration by combining ˜1E7 cells with 10 ug vector (3:1 ratio of each expression vector:vector encoding recombinase) using the Neon electroporator (Invitrogen). Cells recovered in T-75 flasks containing CD-CHO medium (Gibco)+8 mM glutamine for two days. Selection was applied by resuspending the cells in CD-CHO medium+5 ug/ml blasticidin. Upon recovery, cells were scaled up to 125 mL shake flasks and passaged 2-3 times in CD-CHO medium on a 3-day/4-day schedule, then transferred to propriety in house medium for further passaging and sample harvest.

### Sample Preparation

Cells were harvested from day 3 passaging culture for genomic DNA and total RNA preparation. Genomic samples were prepped using DNeasy Blood & Tissue kit (Qiagen) and quantified using nanodrop. One ug of total genomic DNA was digested using Msel restriction enzyme (New England BioLabs) to fragment genomic DNA, RNA samples were prepared using RNeasy mini kit (Qiagen) and quantified using nanodrop.

Two ug total RNA was converted to cDNA using SuperScript™ III First-Strand Synthesis System (ThermoFisher).

### Nucleotide Polymorphism Quantitation

Genomic and cDNA templates were quantified using the QX200™ Droplet Digital™ PCR System (ddPCR) (Bio-Rad). PrimeTime LNA qPCR probes (Integrated DNA Technologies) were used to quantify each nucleotide polymorphism (NP), PrimeTime qPCR probes (Integrated DNA Technologies) were used to detect total heavy chain, total light chain and GAPDH housekeeping gene. PCRs were run in triplicate using standard conditions. Genomic DNA was measured as a ratio of each NP to total heavy and light chain. cDNA was measured as a ratio of total heavy or light chain against the GAPDH housekeeping (endogenous CHO) gene, then multiplied by the decimal percentage of each individual NP based on the ratio of NP to total heavy or light chain.

### Fed-Batch Culture

Pools were subjected to a 12-day fed-batch culturing process using the Ambr®15 instrument. Each pool was seeded at ˜1.5e6 cells/mL run in duplicate vessels using proprietary production and feed media. Pools were evaluated in the Ambr® instrument using Pfizer's platform fedbatch process (see, Lieske, P et al, Biotechnology J, Vol. 15 (4), 1900306, April 2020). Additionally, cell suspension was collected for titer evaluation using HPLC on Days 7, 10, and 12. For multi-specific expressing pools, each vessel was harvested and for selected pools, conditioned medium was submitted for product quality analysis at the conclusion of the run.

### Size Exclusion Chromatography (SEC)

Approximately 50 mg of each protein sample was injected onto a YMC-Pack Diol-200 size-exclusion column (300×8 mm, Waters Catalog number DL20S053008WT) maintained at 30 C. The high molecular mass species (HMMS), monomer, and LMMS were separated using isocratic elution with a mobile phase containing 20 mM sodium phosphate and 400 mM sodium chloride, pH 7.2.

Capillary Gel Electrophoresis (cGE)

Approximately 5 mg of each protein sample was incubated at 70 C for 10 minutes under both reducing (DTT) and non-reducing (iodoacetamide) conditions and analyzed using HT Protein Express chip according to Chen, X et al, Electrophoresis, Vol. 29 (24) 4993-5002, July 2008.

### Results

To assess the accuracy of the ddPCR assay, genomic DNA was harvested from cellular pools from configurations C2 and C3. All 8 NPs (4 heavy and 4 light chain NPs) are represented across these two sets of pools. Since each pool contains 2 heavy and 2 light chain genes, each NP is expected to account for 50% of the total signal for each gene in each pool (e.g. in the C2 pool, NP1 was expected to account for 50% of the LC gene signal, and NP2 was expected to account for 50% LC gene signal). Each NP was found to account for approximately 50% of the total signal for each gene in each pool as expected, confirming the accuracy of the ddPCR assay.

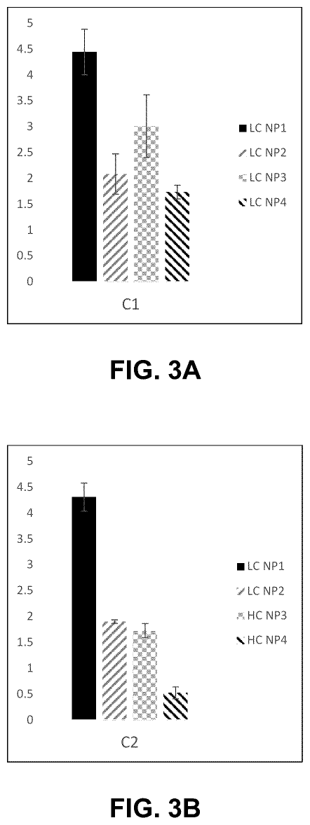

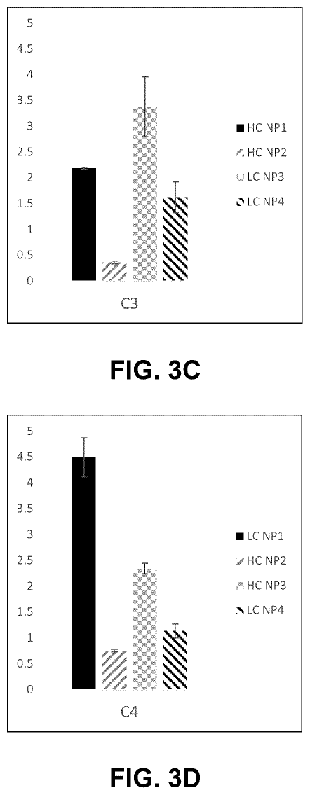

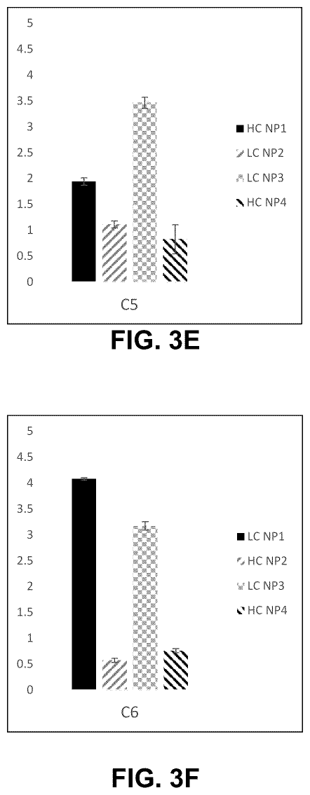

Next, to assess the transcript level for each expression vector position for each of the configurations, RNA was collected from cells and converted to cDNA. NPs, total heavy or light chain and the GAPDH housekeeping gene levels was measured for each sample. Total heavy or light chain was normalized to GAPDH, then multiplied by the decimal percentage for each NP based on the ratio of NP to total heavy or light chain. FIGS. 3A-3F shows the transcript levels contributed from each expression vector position for all configurations tested. Transfection configuration 1 (C1) has light chain occupancy for all expression vector positions, allowing for direct comparison using the same GOI to assess relative strength across all expression vector positions. Light chain was used for this assessment instead of heavy chain, since expression of heavy chain without sufficient light chain can result in cellular toxicity. It was observed that the first position in each expression vector in each of the landing pads is stronger than the second position. It was also seen that the first position of the expression vector occupying landing pad A is stronger than the first position of the expression vector occupying landing pad B. These results show that differences in transcript levels occur based on the position of the transgene within the expression vector, as well as transcriptional differences between each landing pad.

Conditions C2 and C3 introduce heavy chains into either landing pad B or landing pad A, respectively. As compared to C1, there is less heavy chain transcript produced than light chain for all expression vector positions. Results also show that more first position heavy chain transcript is being produced than second position heavy chain for both landing pads, which is consistent with light chain results from C1. Additionally, the light chain transcript levels for C2 and C3 are comparable to that of C1, suggesting the landing pads are independent of each other, such that varying the occupancy of gene inserts in one landing pad will not affect the other. These results show differences in heavy chain and light chain transcription, and that heavy chain transcription results in lower levels of transcript as compared to light chain. However, the observation of first position yielding more transcript than the second position for each of the landing pads is maintained for both the light chain and heavy chain.

Transfection C4 and C5 assess the expression vector positional effect of heavy chain following light chain, and light chain following heavy chain for each landing pad. In both iterations, results show greater transcript levels from position 1 as opposed to position 2 for each of the landing pads, even when heavy chain occupies position 1. When comparing second position light chain levels from C4 and C5 vs. C1, less light chain transcript is observed when first position is occupied by heavy chain as opposed to light chain. Conversely, when comparing second position heavy chain levels from C4 and C5 vs. C2 and C3, more heavy chain is observed when the first position is occupied by light chain rather than heavy chain. These results strongly suggest that the second position transcript level is dependent on the occupancy of the first position of the expression vector. However, when comparing first position heavy and light chain across all conditions, heavy and light chain transcript levels remain consistent regardless of the occupancy of the second position. These results suggest that first position transcript levels are independent of second position occupancy.

Configuration C6 is the vector configuration previously utilized with this CHO expression system (see, Inniss, supra). The results from configuration C6 show that each landing pad is contributing a large surplus of light chain vs. heavy chain transcript. The C6 results suggested that expression productivity might be improved by increasing heavy chain transcript levels and increasing the heavy to light chain transcript ratio.

The data in FIGS. 3A-3F were used to generate a numerical key (Table 3) for predicting estimated transcript levels for all possible expression vector configurations in each landing pad for standard mAbs. This key was generated by taking the average transcript levels observed for each expression vector configuration for each individual landing pad. For Table 3, the average transcript level was quantified as a ratio to housekeeping gene GAPDH (e.g. a value of “4.4” in a position indicates a relative ratio of the transcript level of the transgene in that position to the transcript level of GAPDH) and the relative percentage of light (L) and/or heavy (H) chain for 1^{st} and 2^{nd} expression vector position (relative to the 1^{st} L chain in Landing Pad A in the L/L configuration; see bolded value) in each of the landing pads for all possible occupancy combinations.

TABLE 3

| | 1^{st}/2^{nd} | | | 1st | 2^{nd} |
|---|---|---|---|---|---|
| Landing | Position | 1^{st} | 2^{nd} | Position | Position |
| Pad | Occupancy | Position | Position | % | % |
| A | L/L | 4.4 | 2.0 | 100.0 | 45.4 |
| | L/H | 4.3 | 0.7 | 98.9 | 15.6 |
| | H/L | 1.9 | 1.1 | 44.4 | 25.4 |
| | H/H | 2.2 | 0.4 | 50.0 | 8.2 |
| B | L/L | 3.2 | 1.7 | 72.1 | 38.6 |
| | L/H | 3.3 | 0.8 | 76.5 | 18.2 |
| | H/L | 2.3 | 1.1 | 53.5 | 26.0 |
| | H/H | 1.7 | 0.5 | 39.4 | 11.9 |

Using the information in Table 3, it was predicted that the vector configuration outlined in C2 [herein also referred to as “LL HH”, since the vector for Landing Pad A contains a light chain in position 1 and a light chain in position 2 (“LL”), and the vector for Landing Pad B contains a heavy chain in position 1 and a heavy chain in position 2 (“HH”)] would have a superior transcript profile as compared to the vector configuration C6 (herein referred to as “LH LH”, based on the same rationale as described for C2) and thus improve productivity. This was calculated as follows. For the LL HH (C2) configuration, the total light chain value is: 6.4 [calculated by adding: 4.4 (1^{st} position)+2.0 (2^{nd} position)], and the total heavy chain value is 2.2 [calculated by adding 1.7 (1^{st} position)+0.5 (2^{nd} position)]. Thus, the ratio of light chain to heavy chain transcript in the cell for the C2 (LL HH) configuration is 6.4 to 2.2, or ˜2.9 light chain per 1 heavy chain. For the C6 (LH LH) configuration, the total light chain value is: 7.6 [calculated by adding: 4.3 (1^{st} position, landing pad A)+3.3 (1^{st} position, landing pad B)], and the total heavy chain value is 1.5 [calculated by adding 0.7 (2^{nd} position, landing pad A)+0.8 (2^{nd} position, landing pad B)]. Thus, the ratio of light chain to heavy chain transcript in the cell for the LH LH configuration is 7.6 to 1.5, or ˜5.1 light chain per 1 heavy chain. In sum, this information indicated there were fewer light chain transcripts to heavy chain transcripts in cells having the LL HH (C2) configuration (˜2.9 to 1) than in cells having the LH LH (C6) configuration (˜5.1 to 1).

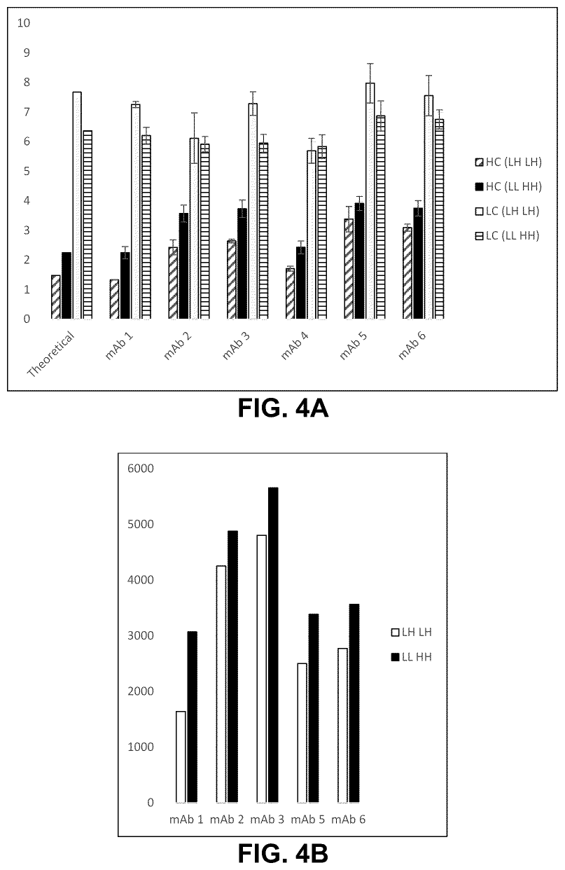

To test configuration C2 versus C6 for the expression of multiple antibodies, both expression vector configurations were generated for six different mAbs (mAb1-mAb6). Each vector was transfected into the CHO host cell line to generate 3 individual pools, followed by selection and recovery. Recovered cells were passaged in shake flasks and samples were harvested for transcript analysis. FIG. 4A shows the heavy and light transcript levels for each vector configuration as predicted by the numerical key in Table 3, compared to the observed transcript levels for all six mAbs. As predicted, the heavy chain transcript level increased significantly for all six mAbs for the LL HH (C2) vector configuration as compared to the LH LH (C6) configuration. Light chain levels decreased for four of the six mAbs, though not all were statistically significant. In all cases however, the heavy to light chain ratio is increased and the overall improvement of the transcript profile resulted in increased mAb productivity of the C2 (LL HH) pools as compared to the C6 (LH LH) pools following 12-day fed-batch culture (FIG. 4B). The persistent pattern of the transcript profile across all six mAbs for each of the vector configurations and the consistent improvement in productivity suggests the learnings from this study can be applied universally to standard mAbs and allows for up-front, rational expression vector design.

### Example 2: Assessment of Order and Positional Effects mAb-Fusion Protein Encoding Genes in Vectors in a Dual-Landing Pad CHO Cell Line

### Goal:

In this example, the effect of location in expression vectors on transcription and titer of three different genes that encode polypeptides of a mAb-fusion protein (antibody heavy chain, antibody light chain, and antibody heavy chain-cytokine fusion) in a dual-landing pad CHO cell line was assessed.

### Materials and Methods

Unless otherwise noted, materials and methods were used as described in Example 1, except genes encoding the polypeptides of the mAb-fusion protein used (instead of the standard mAb heavy chain and light chain genes of Example 1).

The mAb-fusion protein contains 3 different polypeptide species: i) antibody heavy chain (HC), ii) antibody light chain (LC), and iii) antibody heavy chain-cytokine fusion (HC-F) (cytokine covalently linked to the C-terminus of the heavy chain). The ratio of these polypeptide species in the mAb-fusion protein is 1 heavy chain:1 heavy chain-cytokine fusion:2 light chains (1 HC:1 HC-F:2 LC). The mAb fusion protein can also be described as a standard mAb, with a cytokine covalently linked to the N-terminus of one of the heavy chains of the mAb.

Two different expression vector configurations were selected for evaluation, as shown in Table 4.

| | Vector for | | Vector for | |
|---|---|---|---|---|
| | Landing Pad A | | Landing Pad B | |
| Configuration | Position 1 | Position 2 | Position 1 | Position 2 |
| # | Gene | Gene | Gene | Gene |
| C1 | LC | HC | HC-F | HC |
| C2 | LC | LC | HC-F | HC |

Configuration C1 was designed based on information in Table 3 to generate transcript levels that would be close to the desired is 1 heavy chain (HC):1 heavy chain-cytokine fusion (HC-F):2 light chains (LC) ratio. Configuration C2 was designed based the information in Table 3, but also to take into account potential differences in translation and/or stability rates for large heavy-chain cytokine fusion polypeptides as compared to standard heavy chain polypeptides, and thus aimed to generate a surplus of HC-F transcript versus HC transcript to compensate for expected reduced translation and/or stability of HC-F as compared to HC.

### Results

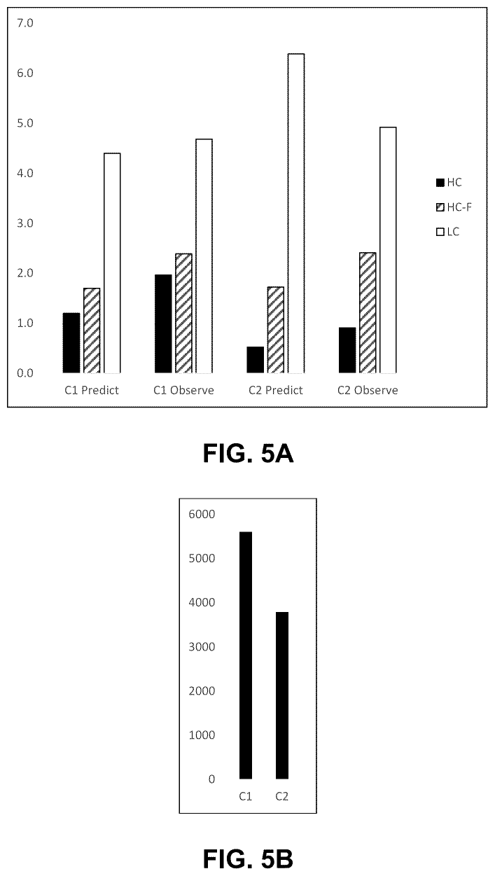

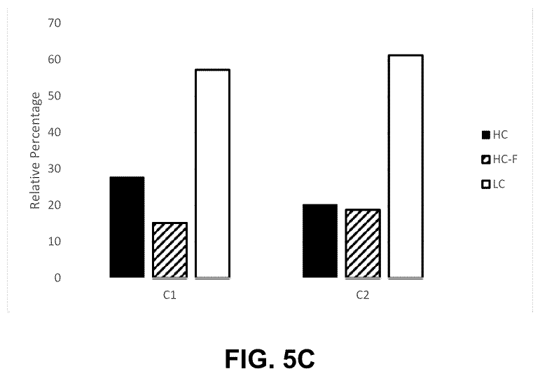

The predicted and observed transcript level for each vector configuration are shown in FIG. 5A. As shown in FIG. 5A, the observed transcript level for all genes in both configuration 1 (C1) and configuration 2 (C2) closely matched the predicted transcript level for the genes in the respective configurations. Specifically, vector configuration C1 very closely achieved the predicted 1 HC: 1 HC-F:2 LC ratio of transcript, while configuration C2 shows the predicted surplus of HC-F transcript as compared to HC.

Antibody titer from the cells was also assessed. Good productivity was seen for both configurations following 12-day fed-batch, with configuration C1 showing higher productivity than C2 (˜5500 mg/L versus ˜3800 mg/L, respectively)(FIG. 5B).

Polypeptide levels measured via densitometry of reduced SDS-PAGE analysis show an excess of HC vs. HC-F for configuration C1, and roughly equal levels of HC and HC-F for configuration C2 (FIG. 5C), suggesting reduced translation rate/stability for the larger HC-F polypeptide. Next, product quality analysis was performed on the C1 and C2 products via analytical size chromatography (“SEC”) and capillary gel electrophoresis (“cGE”). The results are shown in Table 5.

TABLE 5

| | | | | % Intact (cGE assay) | |
|---|---|---|---|---|---|
| | SEC Assay | | | Non- | |
| | % HMMS | % POI | % LMMS | reduced | Reduced |
| C1 | 1.96 | 50.58 | 47.46 | 34.6 | 96.1 |
| C2 | 0.66 | 98.79 | 0.55 | 81.4 | 97.1 |

The data in FIG. 5A and Table 5 showed that while configuration C1 had the transcript ratio closest to the targeted 1 HC:1 HC-F:2 LC ratio (FIG. 5A), the reduced translational rate and/or stability of the HC-F polypeptide resulted in a surplus of HC polypeptide, which presumably led to the increased percentage of low molecular mass species (“LMMS”) in the C1 product (Table 5). In contrast, the product from configuration C2 showed a superior quality profile (Table 5) as evidenced by, for example, the much higher % protein of interest (POI)(the POI is the mAb-fusion protein) and % intact protein, and much lower % LMMS in C2 as compared to C1, showing that most of the C2 product is intact mAb-fusion protein, while nearly half of the product in C1 is LMMS. The superior quality profile for the product from the C2 configuration is likely due to the presence of similar levels of the HC and HC-F polypeptides (FIG. 5C).

Overall, the data in this Example shows the benefits of the C2 vector configuration (LC-LC/HC-F-HC) for the production of a mAb-fusion protein as part of a dual expression vector-based protein expression system.

Although the disclosed teachings have been described with reference to various applications, methods, kits, and compositions, it will be appreciated that various changes and modifications can be made without departing from the teachings herein and the claimed invention below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not

1. A mammalian host cell comprising a first exogenous nucleic acid construct integrated into a host cell chromosome and a second exogenous nucleic acid construct integrated into a host cell chromosome, wherein the first exogenous nucleic acid construct comprises in 5′ to 3′ order a first gene encoding an antibody light chain and a second gene encoding an antibody light chain, and wherein the second exogenous nucleic acid construct comprises in 5′ to 3′ order a first gene encoding an antibody heavy chain or antibody heavy chain fusion protein and a second gene encoding an antibody heavy chain.

2. A mammalian host cell comprising a first exogenous nucleic acid construct integrated into a host cell chromosome and a second exogenous nucleic acid construct integrated into a host cell chromosome,
wherein the first exogenous nucleic acid construct comprises the following elements in 5′ to 3′ order: a) first recombination sequence; b) first gene encoding an antibody light chain; c) second gene encoding an antibody light chain; d) second recombination sequence, and
wherein the second exogenous nucleic acid construct comprises the following elements in 5′ to 3′ order: a) first recombination sequence; b) first gene encoding an antibody heavy chain or antibody heavy chain fusion protein; c) second gene encoding an antibody heavy chain; d) second recombination sequence.

3-4. (canceled)

5. The host cell of claim 2, wherein the first recombination sequence of the first exogenous nucleic acid construct has a different nucleotide sequence than the second recombination sequence of the first exogenous nucleic acid construct by at least one nucleotide.

6. The host cell of claim 5, wherein the first recombination sequence of the first exogenous nucleic acid construct and the first recombination sequence of the second exogenous nucleic acid construct are recognized by the same recombinase enzyme.

7. The host cell of claim 5, wherein the first recombination sequence of the first exogenous nucleic acid construct, the second recombination sequence of the first exogenous nucleic acid construct, the first recombination sequence of the second exogenous nucleic acid construct, and the second recombination sequence of the second exogenous nucleic acid construct are recognized by the same recombinase enzyme.

8. The host cell of claim 2, wherein the first recombination sequence of the first exogenous nucleic acid construct has the same nucleotide sequence as the first recombination sequence of the second exogenous nucleic acid construct.

9. The host cell of claim 2, wherein the first recombination sequence of the first exogenous nucleic acid construct has a different nucleotide sequence than the first recombination sequence of the second exogenous nucleic acid construct by at least one nucleotide.

10. The host cell of claim 2, wherein the first recombination sequence of the first exogenous nucleic acid construct, the second recombination sequence of the first exogenous nucleic acid construct, the first recombination sequence of the second exogenous nucleic acid construct, and the second recombination sequence of the second exogenous nucleic acid construct each differ from each other by at least one nucleotide.

11. The host cell of claim 2, wherein the first recombination sequence is recognized by a tyrosine site-specific recombinase or a serine site specific recombinase.

12. The host cell of claim 11, wherein the tyrosine site specific-recombinase is Cre recombinase or Flp recombinase.

13. The host cell of claim 11, wherein the serine site specific recombinase is Bxb1 recombinase.

14. The host cell of claim 1, wherein the cell is a mouse cell, a rat cell, a Chinese Hamster Ovary (CHO) cell, or a human cell.

15. (canceled)

16. A method for producing an antibody or antibody fusion protein, the method comprising:
(a) providing the mammalian host cell of claim 1,
(b) culturing the host cell under conditions sufficient to express the antibody or antibody fusion protein.

17-19. (canceled)

20. A composition comprising a first expression vector and a second expression vector,
wherein the first expression vector comprises the following elements in 5′ to 3′ order: a) first recombination sequence; b) first gene encoding an antibody light chain; c) second gene encoding an antibody light chain; d) second recombination sequence, and
wherein the second expression vector comprises the following elements in 5′ to 3′ order: a) first recombination sequence; b) first gene encoding an antibody heavy chain or antibody heavy chain fusion protein; c) second gene encoding an antibody heavy chain; d) second recombination sequence.

21-22. (canceled)

23. The composition of claim 20, wherein the first recombination sequence of the first expression vector has a different nucleotide sequence than the second recombination sequence of the first expression vector by at least one nucleotide.

24. The composition of claim 23, wherein the first recombination sequence of the first expression vector and the first recombination sequence of the second expression vector are recognized by the same recombinase enzyme.

25. The composition of claim 23, wherein the first recombination sequence of the first expression vector, the second recombination sequence of the first expression vector, the first recombination sequence of the second expression vector, and the second recombination sequence of the second expression vector are recognized by the same recombinase enzyme.

26. The composition of claim 20, wherein the first recombination sequence of the first expression vector has the same nucleotide sequence as the first recombination sequence of the second expression vector.

27. (canceled)

28. The composition of claim 20, wherein the first recombination sequence of the first expression vector, the second recombination sequence of the first expression vector, the first recombination sequence of the second expression vector, and the second recombination sequence of the second expression vector each differ from each other by at least one nucleotide.

29. The composition of claim 20, wherein the first recombination sequence is recognized by a tyrosine site-specific recombinase or a serine site specific recombinase.

30-32. (canceled)
